(19) United States
(12) Patent Application Publication
(10) Pub. No.: US 2025/0197322 A1
(43) Pub. Date: Jun. 19, 2025

(54) PROCESSES AND APPARATUSES FOR REMOVING CARBON MONOXIDE FROM AN ETHYLENE STREAM

(71) Applicant: UOP LLC, Des Plaines, IL (US)

(72) Inventors: Kyle E. Krynski, Chicago, IL (US); Anh Ngo, Carol Stream, IL (US); Steven E. Zell, Salt Lake City, UT (US); Michael Pierce, Erie, CO (US); Rajasi Shukre, Wheeling, IL (US)

(21) Appl. No.: 18/919,072

(22) Filed: Oct. 17, 2024

Related U.S. Application Data
(60) Provisional application No. 63/611,445, filed on Dec. 18, 2023.

(51) Int. Cl. C07C 7/04; C07C 1/24; C07C 7/00; C07C 7/09; C07C 7/11

(52) U.S. Cl. CPC C07C 7/04; C07C 1/24; C07C 7/005; C07C 7/09; C07C 7/11

(57) ABSTRACT

Processes and apparatuses for separating carbon monoxide from ethylene. The ethylene may be produced from biobased alcohol. The separation utilizes a fractionation column which produces a carbon monoxide depleted ethylene stream and an overhead stream comprising carbon monoxide and ethylene. The ethylene from the overhead stream may be recovered in an absorption zone with an absorbing liquid. A oxygenate fractionation zone may be utilized to remove oxygenates from the carbon monoxide depleted ethylene stream.

### RELATED APPLICATIONS

This application claims the benefit of U.S. Provisional Patent Application Ser. No. 63/611,445 filed on Dec. 18, 2023, the entire disclosure of which is incorporated herein by way of reference.

### FIELD OF THE INVENTION

This invention relates generally to processes and apparatuses for separating carbon monoxide from an ethylene stream, and more particular to removing carbon monoxide from ethylene streams produced during the production of jet fuel

### BACKGROUND OF THE INVENTION

As the demand for fuel increases worldwide, there is increasing interest in producing fuels and blending components from sources other than crude oil. Often referred to as a biorenewable source, these sources include, but are not limited to, plant oils such as corn, rapeseed, canola, soybean, microbial oils such as algal oils, animal fats such as inedible tallow, fish oils and various waste streams such as yellow and brown greases and sewage sludge. A common feature of these sources is that they are composed of glycerides and free fatty acids (FFA). Both triglycerides and the FFAs contain aliphatic carbon chains having from about 8 to about 24 carbon atoms. The aliphatic carbon chains in triglycerides or FFAs can be fully saturated, or mono, di or poly-unsaturated.

One such exemplary process is the production of sustainable aviation fuel (SAF) from biorenewable source, such as biorenewable derived ethanol. The conversion of ethanol feedstock to SAF is generally completed in four chemical processing steps: dehydration, oligomerization, hydrogenation, and fractionation. In such a processing scheme, carbon monoxide may negatively impact the oligomerization. Thus, it may be desirable to separate the carbon monoxide from the ethylene before the oligomerization step.

One technique for removing carbon monoxide impurity from ethylene is to pass the ethylene over a bed of solid adsorbent particles. The adsorbent material to selectively captures carbon monoxide while allowing ethylene molecules to pass through without hinderance.

There is an ongoing need for more ways to separate carbon monoxide from ethylene.

### SUMMARY OF THE INVENTION

The present inventors have discovered new processes and apparatus for separating carbon monoxide from ethylene.

The disclosed invention applies the concepts of fractionation at high pressures and cold temperatures to separate carbon monoxide from ethylene. In the present processes, the ethylene process stream is used as a significant source of cooling in the design via flashing and auto-refrigeration, which provides more efficient processes.

Additionally, the present invention also reduces ethylene loss with absorption zone using an absorbing liquid. The absorbing liquid may be an existing process slipstream from within the oligomerization process.

The present processes provide a reduction of carbon monoxide in ethylene to low ppm levels (˜5-50 vppm). At the same time, ethylene losses due to carbon monoxide removal processing may be minimized (<0.01%).

It has been surprisingly found that cold fractionation of ethylene and carbon monoxide provide efficient processes. In general, the cryogenic purification of ethylene at extreme cold temperatures has high operating costs. This is considered undesirable for insertion into SAF production processes due to these high costs. Additionally, such purification of ethylene would be thought to increase the carbon intensity of SAF production and make it less sustainable. The present inventors have discovered otherwise.

Therefore, the present invention may be characterized, in at least one aspect, as providing a process for separating carbon monoxide from ethylene by: cooling an ethylene stream with a refrigerant stream in a cooling zone; separating the ethylene stream in a fractionation zone comprising a fractionation column into a liquid stream comprising ethylene and an overhead stream comprising carbon monoxide and ethylene; separating the overhead stream in a vessel into a liquid portion and a gaseous portion, wherein the gaseous portion comprises carbon monoxide and ethylene; and, absorbing the ethylene from the gaseous portion in an absorption zone with an absorbing liquid stream to provide an enriched absorbing liquid comprising an increased level of ethylene and an ethylene depleted vapor comprising carbon monoxide.

The process may also include reboiling a stream from the fractionation column with the ethylene stream before cooling the ethylene stream in the cooling zone.

The process may include heating the liquid stream from the fractionation column with the ethylene stream before cooling the ethylene stream in the cooling zone.

The process may include cooling the overhead stream with a refrigerant stream in a cooling zone before separating the overhead stream in a vessel.

The process may include subcooling the refrigerant stream by transferring heat from the refrigerant stream to a portion of the liquid stream from the fractionation column.

The process may further include subcooling the refrigerant stream by transferring heat from the refrigerant stream to the gaseous portion from the vessel.

The absorbing liquid stream may be a hydrocarbon stream. The hydrocarbon stream may be an oligomerized effluent. The enriched absorbing liquid may be reboiled.

The ethylene stream may be a portion of a dehydration effluent.

The ethylene stream may have a pressure of between 3,447 to 4,137 kPa (500 to 600 psi(g)) and the liquid stream may have a pressure between 2,413 to 3,103 kPa (350 to 500 psi(g)).

The process may include recovering power from a pressure decrease from the ethylene depleted vapor with an expansion device.

The process may include cooling a process stream with the liquid stream.

The process may include separating the liquid ethylene stream in a oxygenate fractionation zone comprising a fractionation column into a liquid stream comprising oxygenates and a oxygenate fractionation zone overhead stream and, separating the oxygenate fractionation zone overhead stream in a vessel into a liquid portion and a gaseous portion, wherein the gaseous portion comprises ethylene and carbon dioxide. The process may further include reboiling a stream from the fractionation column in the oxygenate fractionation zone with a hot process stream. The process may additionally include cooling the oxygenate fractionation zone overhead stream with a refrigerant stream in a cooling zone before separating the overhead stream in the vessel.

The present invention may also be generally characterized as providing a process for producing jet range hydrocarbons from a biobased alcohol by: dehydrating a biobased alcohol stream in a dehydration zone comprising a reactor with a catalyst and being operated under conditions to provide a dehydrated effluent, the dehydrated effluent comprising ethylene and carbon monoxide; separating, from a feed stream comprising a portion of the dehydrated effluent, the carbon monoxide from the ethylene in a fractionation zone, the fractionation zone providing a bottoms stream comprising a carbon monoxide depleted ethylene stream and an overhead stream comprising carbon monoxide and ethylene; absorbing ethylene from a portion of the overhead stream in an absorption zone with an absorbing liquid stream to provide an enriched absorbing liquid stream comprising an increased level of ethylene and an ethylene depleted vapor stream comprising carbon monoxide; oligomerizing the carbon monoxide depleted liquid ethylene stream in an oligomerizing zone comprising a reactor with a catalyst and being operated under conditions to provide an oligomerized effluent; hydrogenating the oligomerized effluent in a hydrogenation zone having a hydrogenation reactor with a catalyst and being operated under conditions to provide a hydrogenated effluent; and, separating the hydrogenated effluent into one or more hydrocarbon streams, the one or more hydrocarbon streams comprising a jet fuel hydrocarbon steam.

The absorbing liquid stream may be the oligomerized effluent.

The process may include separating the overhead stream in a vessel into a liquid portion and a gaseous portion, wherein the gaseous portion comprises the portion of the overhead stream from which ethylene is absorbed in the absorption zone, or cooling the overhead stream with a refrigerant stream in a cooling zone before separating the overhead stream; or both.

The process may include separating the oxygenates from the carbon monoxide depleted ethylene stream in a oxygenate fractionation zone comprising a fractionation column into a liquid stream comprising oxygenates and an oxygenate fractionation zone overhead stream before oligomerizing the carbon monoxide depleted ethylene stream.

Additional aspects, embodiments, and details of the invention, all of which may be combinable in any manner, are set forth in the following detailed description of the invention.

### DETAILED DESCRIPTION OF THE DRAWINGS

One or more exemplary embodiments of the present invention will be described below in conjunction with the following drawing figures, in which:

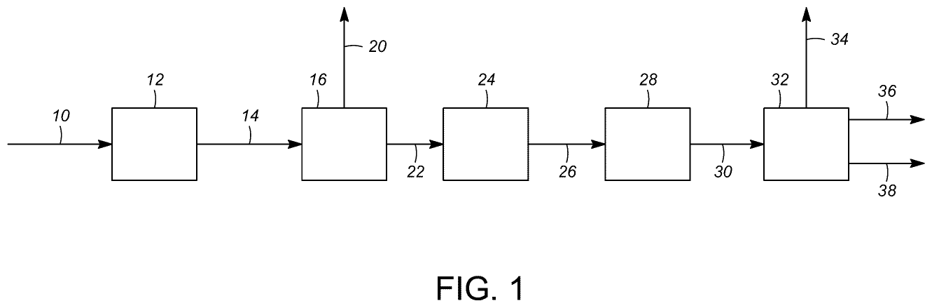

FIG. 1 shows a process flow diagram according to one or more embodiments of the present invention; and,

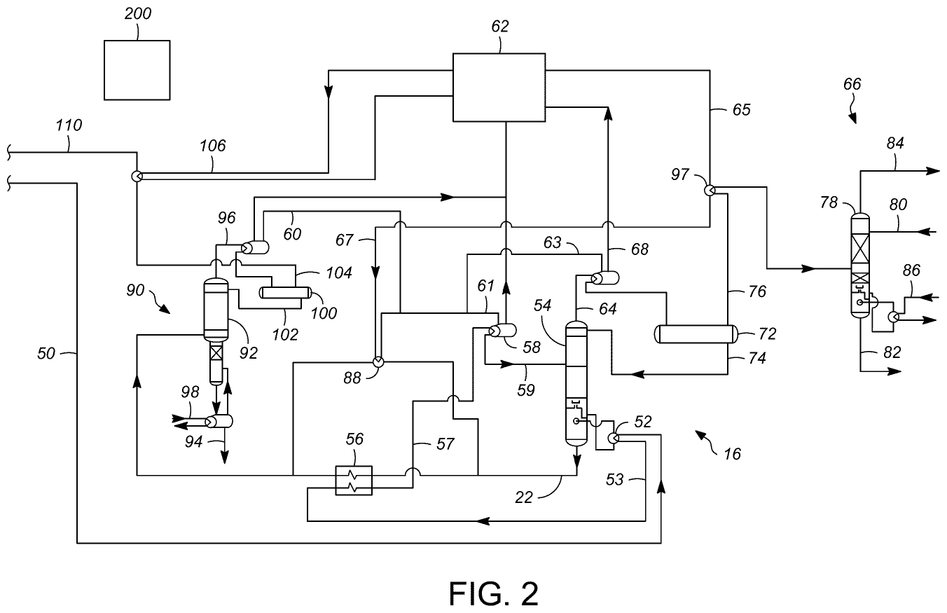

FIG. 2 shows a process flow diagram of a fractionation zone according to one or more embodiments of the present invention.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

### DETAILED DESCRIPTION OF THE INVENTION

As mentioned above, processes and apparatuses for separating carbon monoxide from ethylene have been invented. The present processes separate carbon monoxide from bulk ethylene via cold fractionation (low temperature, high pressure). Additionally, the present processes also minimize the loss of ethylene via selective recovery with a sponge absorber system. The present invention also allows for effective and efficient heat integration with process and utility systems. While these methods find greatest utility in converting feedstocks from alkanols, thereby allowing for production of jet fuels from renewable sources, this is not intended to limit the application of the methods of the present invention.

With these general principles in mind, one or more embodiments of the present invention will be described with the understanding that the following description is not intended to be limiting.

With reference to FIG. 1, a biobased alcohol stream 10 is passed to a first reaction zone 12. The biobased alcohol stream 10 is preferably, a biobased ethanol stream. Accordingly, the following detailed description will proceed with the embodiment that the feed stream 10 comprises a biobased ethanol stream however this is merely illustrative.

Additionally, “bio-based” refers to organic materials in which the carbon comes from recently (on the order of centuries) fixated carbon dioxide present in the atmosphere using sunlight energy (photosynthesis). On land, this carbon dioxide is captured or fixated by plant life (e.g., agricultural crops or forestry materials). In the oceans, the carbon dioxide is captured or fixated by photosynthesizing bacteria or phytoplankton. For example, a bio-based material has a ¹⁴C/¹²C isotope ratio greater than zero. Contrarily, a fossil-based material has a ¹⁴C/¹²C isotope ratio of zero. The term “renewable” with regard to compounds such as alcohols or hydrocarbons (olefins, di-olefins, polymers, etc.) also refers to compounds prepared from biomass using thermochemical methods (e.g., Fischer-Tropsch catalysts), biocatalysts (e.g., fermentation), or other processes, for example as described herein.

Biomass fermentation products typically include lower isoalkanols such as, for example, C₂ to C₈ isoalkanols obtained by contacting biomass with biocatalysts that facilitate conversion (by fermentation) of the biomass to isoalkanols. The biomass feedstock for such fermentation processes can be any suitable fermentable feedstock known in the art, such as fermentable sugars derived from agricultural crops including sugarcane, corn, etc. Suitable fermentable biomass feedstock can also be prepared by the hydrolysis of biomass, for example lignocellulosic biomass (e.g. wood, corn stover, switchgrass, herbiage plants, ocean biomass, etc.), to form fermentable sugars.

In FIG. 1, the first reaction zone 12 is a dehydration zone comprising a reactor with a catalyst and being operated under conditions to provide a dehydrated effluent 14 which includes ethylene and carbon monoxide and which may also include hydrogen, carbon dioxide, methane, ethane, propane, propylene, butane, butenes, pentane, pentenes, and oxygenates such as diethyl ether, diethoxyethane, and ethyl acetate.

Suitable catalyst may include alumina, modified alumina, silicoaluminate, modified silicoaluminate, or other catalysts known in the art. The reactor may be operated at a temperature from 200° to 500° C. (392° to 932° F.). In some embodiments, the dehydration reactor may be operated at a pressure from 0 to 8,300 kPa (0 to 1, 204 psi(g)). In some embodiments, the dehydration reactor may be operated at a pressure from 0 to 3,500 kPa (0 to 508 psi(g)). In addition to the biobased alcohol stream, an insert gas such as nitrogen or steam may be introduced to the first reaction zone 12.

As noted above, the presence of the carbon monoxide in the dehydrated effluent 14 may be undesirable for downstream processes, accordingly, in the present processes, the dehydrated effluent 14, or a portion thereof, forms a feed stream for a fractionation zone 16. The fractionation zone 16 is described in more detail below. However, generally, the fractionation zone 16 includes a fractionation column and provides a carbon monoxide depleted ethylene stream 22 and ethylene depleted vapor stream 20 comprising a significant portion of the carbon monoxide from the dehydrated effluent 14.

The carbon monoxide depleted ethylene stream 22 is passed to an oligomerizing zone 24 comprising a reactor with a catalyst and being operated under conditions to provide an oligomerized effluent 26.

In the oligomerizing zone 24, the ethylene is converted into a mixture of heavier boiling hydrocarbons including jet range hydrocarbons via oligomerization by reacting the olefins using a catalyst under appropriate conditions. For example, the oligomerization zone 24 may, without limitation, be operated at a temperature from about 100 to about 300° C. (212 to 572° F.) and a pressure of from about 689 to about 6,895 kPa (100 to 1,000 psi(g)).

The oligomerization catalyst in the oligomerization zone 24 is not limited to any particular catalyst and may comprise any catalyst suitable for catalyzing conversion of the one or more biorenewable C₂ to C₈ olefins in the olefin stream to olefinic oligomers comprising heavier boiling C₅₊ hydrocarbons, including jet-range hydrocarbons. The oligomerization catalyst may be any such catalyst known now or in the future. Exemplary oligomerization catalysts are described in U.S. Pat. Pub. No. 2023/0313048.

As shown in FIG. 1, the oligomerized effluent 26 from the oligomerization zone 24 may be passed to a hydrogenation zone 28 having a hydrogenation reactor with a catalyst and being operated under conditions to provide a hydrogenated effluent 30.

Hydrogenation is typically performed using a conventional hydrogenation or hydrotreating catalyst, which may include metallic catalysts containing, e.g., palladium, rhodium, nickel, ruthenium, platinum, rhenium, cobalt, molybdenum, or combinations thereof, and the supported versions thereof. Catalyst supports can be any solid, inert substance including, but not limited to, oxides such as silica, alumina, titania, calcium carbonate, barium sulfate, and carbons. The catalyst support can be in the form of powder, granules, pellets, or the like. Hydrogenation suitably occurs at a temperature of between 38° to 260° C. (100° to 500° F.) and at a pressure of between about 689 to about 6,895 kPa (100 to 1,000 psi(g)). Other process conditions known by those of ordinary skill in the art may be utilized.

The hydrogenated effluent 30 from the hydrogenation zone 28 will substantially comprise saturated hydrocarbons (i.e., paraffins). The hydrogenated effluent 30 may be passed to a separation zone 32 having one or more columns configured and operated to separate the hydrogenated effluent 30 into one or more hydrocarbon streams 34, 36, 38 - - - one of which is a jet fuel hydrocarbon steam.

As used herein the term “jet-range hydrocarbons,” “jet-range paraffins,” “jet-range fuels,” or “jet fuels” can include hydrocarbons having a boiling point temperature in the range of about 130 to about 300° C. (266 to 572° F.), preferably 150 to 260° C. (302 to 500° F.), at atmospheric pressure. Additionally, as used herein, the terms “jet-range hydrocarbons,” “jet-range paraffins,” “jet-range fuels,” or “jet fuels” refer to a mixture of primarily C₈ to C₁₆ hydrocarbons with a freezing point of about −40° C. (−40° F.) or about −47° C. (−52.6° F.).

Turning to FIG. 2, as discussed above, the present invention utilizes the fractionation zone 16 to separate carbon monoxide and ethylene in an ethylene feed stream 50. The ethylene feed stream 50 comprises a portion of the 14 (FIG. 1). Although not depicted as such, the ethylene feed stream 50 may be first compressed in various compressors and dried in a drying section of the 12 (FIG. 1) to remove water before fractionation. The ethylene feed stream 50 may have a pressure of between 3,447 to 4,137 kPa (500 to 600 psi(g)) or about 3,758 kPa (545 psi(g)).

In order to remove heat from the ethylene feed stream 50, the ethylene feed stream 50 may first pass to a reboiler 52 to reboil a stream from the fractionation column 54 in the fractionation zone 16. From the reboiler 52, a cooled, ethylene feed stream 53 may be passed to a condenser 56 to transfer heat to the carbon monoxide depleted ethylene stream 22 from the fractionation column 54. Further, a cooled, condensed ethylene feed stream 57 may be cooled in a cooling zone 58 receiving a refrigerant stream 61 from a refrigeration unit 62. After passing through the cooling zone 58, the ethylene feed 59, having a temperature between, for example, −26° to −1° C. (−15° to 30° F.) may be passed to the fractionation column 54. In the fractionation column 54, the components of the ethylene feed stream 50 will separate into the carbon monoxide depleted ethylene stream 22 and an overhead stream 64 which includes carbon monoxide and ethylene.

As will be appreciated, the carbon monoxide depleted ethylene stream 22 will primarily be ethylene and may include a small fraction of carbon dioxide, methane, ethane, propane, propylene, butane, butenes, pentane, pentenes, and oxygenates (diethyl ether, diethoxyethane, and ethyl acetate). The carbon monoxide depleted ethylene stream 22 is liquid and has a pressure between 2,413 to 3,447 kPa (350 to 500 psi(g)) for example about 3,103 kPa (450 psi(g)) and a temperature between −18° to 10° C. (0° to 50° F.). Additionally, the overhead stream 64, in addition to carbon monoxide and ethylene, may also include hydrogen and methane. Since the overhead stream 64 includes ethylene, it is desirable to recover the ethylene. Accordingly, the present invention contemplates an absorption zone 66.

As depicted in FIG. 2, before being passed to the absorption zone 66, the overhead stream 64 may be cooled in a cooling zone 68, which receives a refrigerant stream 63 from a refrigerant unit 62, before being passed to a separation vessel 72. In the separation vessel 72, the components of the overhead stream 64 separate and into a liquid portion 74 and a gaseous portion 76. The liquid portion 74, being mostly ethylene, may be pumped back to the fractionation column 54. The gaseous portion 76 will include carbon monoxide and ethylene which is recovered in the absorption zone 66.

Before the gaseous portion 76 is passed to the absorption zone 66, this stream 76 may be used for subcooling a refrigerant stream 65 by transferring heat from the refrigerant stream 65 to the gaseous portion 76 in a cooling zone 97. The gaseous portion 76, having a temperature between −1° to 93° C. (30° to 200° F.) may be passed to an absorber vessel 78 which also receives an absorbing liquid stream 80.

The gaseous portion 76 is passed into a lower portion of the vessel 78, and the absorbing liquid stream 80 is passed into an upper portion of the vessel 78. As the liquid flows downward within the vessel 78 and the vapor flows upward, ethylene in the vapor will be absorbed by the liquid. Accordingly, the absorption zone 66 will provide an enriched absorbing liquid 82 comprising an increased level of ethylene and an ethylene depleted vapor 84 comprising carbon monoxide. The ethylene depleted vapor 84 will contain a majority of the carbon monoxide that has been separated from the ethylene feed stream 50. The ethylene depleted vapor 84 may be used as fuel gas, for example, in the hydrogenation zone 28 (see, FIG. 1).

The absorbing liquid 80 should have a high affinity for ethylene, and a low affinity for carbon monoxide. Accordingly, the absorbing liquid 80 may be a hydrocarbon stream. It is contemplated, for example, that the absorbing liquid is an oligomerization effluent, and preferably, a first stage or second stage oligomerization effluent stream so that the enriched absorbing liquid 82 is returned to the oligomerization zone 24 to increase the yield of SAF. To enhance the rejection of carbon monoxide in the enriched absorbing liquid, the enriched absorbing liquid may be reboiled with a hot stream 86.

Returning the fractionation column 54, the carbon monoxide depleted ethylene stream 22, or a portion thereof, may be utilized for subcooling a refrigerant stream 67 by transferring heat, in a heat exchange zone 88, from the refrigerant stream 67 to carbon monoxide depleted ethylene stream 22. Additionally, as mentioned above, the carbon monoxide depleted ethylene stream 22 may be passed to the condenser 56. It is further contemplated that the carbon monoxide depleted ethylene stream 22 may be used to cool any other process stream. For example, the carbon monoxide depleted ethylene stream 22 may be used to cool a stream in a drier section of the dehydration zone 12. This is merely one contemplated example, and other streams could be cooled by the carbon monoxide depleted ethylene stream 22.

The carbon monoxide depleted ethylene stream 22 has a reduced level of carbon monoxide and therefore may returned to a compression zone and processed further as discussed above in regard to FIG. 1. However, as noted above, the carbon monoxide depleted ethylene stream 22 may also include oxygenates. Accordingly, in some cases it may be desirable to remove these oxygenates. Additionally, the conditions of the carbon monoxide depleted ethylene stream 22 are ideal for oxygenate removal. Therefore, an oxygenate fractionation zone 90 may be provided as depicted in FIG. 2.

The oxygenate fractionation zone 90 includes a column 92 that receives the carbon monoxide depleted ethylene stream 22 which is operated under conditions to separate a liquid stream 94 comprising the oxygenates and a oxygenate fractionation overhead stream 96 which includes ethylene. The column 92 may be reboiled with steam or a hot process stream 98.

The oxygenate fractionation overhead stream 96 may be cooled with a refrigerant stream 60 and then passed to a vessel 100. In the vessel 100, the components will separate into a liquid portion 102, which may be pumped back to the oxygenate fractionation column 92, and a gaseous portion 104 that includes ethylene and, for example, carbon dioxide. Heat may be transferred to the gaseous portion 104 from a stream 106 from the refrigerant unit 62. The gaseous portion 104 is also a carbon monoxide depleted ethylene stream 110, having a pressure between 2,413 to 3,447 kPa (350 to 500 psi(g)) and a temperature between −7° to 66° C. (20° to 150° F.) which may returned to a compression zone and processed further as discussed above.

In general, the present invention provides effective and efficient processes and apparatuses to remove carbon monoxide from ethylene. In order to increase power recovery, it is contemplated that an expansion device, like a turbine, be utilized instead of a valve so as to recover power from the associated pressure drop.

The systems and devices described herein may include a controller 200 or a computing device comprising a processing and a memory which has stored therein computer-executable instructions for implementing the processes described herein. The processing unit may comprise any suitable devices configured to cause a series of steps to be performed so as to implement the method such that instructions, when executed by the computing device or other programmable apparatus, may cause the functions/acts/steps specified in the methods described herein to be executed. The processing unit may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory may be any suitable known or other machine-readable storage medium. The memory may comprise non-transitory computer readable storage medium such as, for example, but not limited to, an electronic, magnetic, optical, electromagnetic, infrared, or semiconductor system, apparatus, or device, or any suitable combination of the foregoing. The memory may include a suitable combination of any type of computer memory that is located either internally or externally to the device such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory may comprise any storage means (e.g., devices) suitable for retrievably storing the computer-executable instructions executable by processing unit.

The methods and systems described herein may be implemented in a high-level procedural or object-oriented programming or scripting language, or a combination thereof, to communicate with or assist in the operation of the controller or computing device. Alternatively, the methods and systems described herein may be implemented in assembly or machine language. The language may be a compiled or interpreted language. Program code for implementing the methods and systems described herein may be stored on the storage media or the device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein.

Computer-executable instructions may be in many forms, including modules, executed by one or more computers or other devices. Generally, modules include routines, programs, objects, components, data structures, etc., that perform particular tasks or implement particular abstract data types. Typically, the functionality of the modules may be combined or distributed as desired in various embodiments.

It will be appreciated that the systems and devices and components thereof may utilize communication through any of various network protocols such as TCP/IP, Ethernet, FTP, HTTP and the like, and/or through various wireless communication technologies such as GSM, CDMA, Wi-Fi, and WiMAX, is and the various computing devices described herein may be configured to communicate using any of these network protocols or technologies.

### EXPERIMENTS

In a simulation of a process according to the present invention, a reduction of carbon monoxide of at least 97% was achieved with less than 0.2% loss of ethylene, with lower utility costs.

### SPECIFIC EMBODIMENTS

While the following is described in conjunction with specific embodiments, it will be understood that this description is intended to illustrate and not limit the scope of the preceding description and the appended claims.

A first embodiment of the invention is a process for separating carbon monoxide from ethylene, the process comprising cooling an ethylene stream with a refrigerant stream in a cooling zone; separating the ethylene stream in a fractionation zone comprising a fractionation column into a liquid stream comprising ethylene and an overhead stream comprising carbon monoxide and ethylene; separating the overhead stream in a vessel into a liquid portion and a gaseous portion, wherein the gaseous portion comprises carbon monoxide and ethylene; and, absorbing the ethylene from the gaseous portion in an absorption zone with an absorbing liquid stream to provide an enriched absorbing liquid comprising an increased level of ethylene and an ethylene depleted vapor comprising carbon monoxide. An embodiment of the invention is one, any or all of prior embodiments in this paragraph up through the first embodiment in this paragraph, further comprising reboiling a stream from the fractionation column with the ethylene stream before cooling the ethylene stream in the cooling zone. An embodiment of the invention is one, any or all of prior embodiments in this paragraph up through the first embodiment in this paragraph, further comprising heating the liquid stream from the fractionation column with the ethylene stream before cooling the ethylene stream in the cooling zone. An embodiment of the invention is one, any or all of prior embodiments in this paragraph up through the first embodiment in this paragraph, further comprising cooling the overhead stream with a refrigerant stream in a cooling zone before separating the overhead stream in a vessel. An embodiment of the invention is one, any or all of prior embodiments in this paragraph up through the first embodiment in this paragraph, further comprising subcooling the refrigerant stream by transferring heat from the refrigerant stream to a portion of the liquid stream from the fractionation column. An embodiment of the invention is one, any or all of prior embodiments in this paragraph up through the first embodiment in this paragraph, further comprising subcooling the refrigerant stream by transferring heat from the refrigerant stream to the gaseous portion from the vessel. An embodiment of the invention is one, any or all of prior embodiments in this paragraph up through the first embodiment in this paragraph, wherein the absorbing liquid stream comprises a hydrocarbon stream. An embodiment of the invention is one, any or all of prior embodiments in this paragraph up through the first embodiment in this paragraph, wherein the hydrocarbon stream comprises an oligomerized effluent. An embodiment of the invention is one, any or all of prior embodiments in this paragraph up through the first embodiment in this paragraph, wherein the enriched absorbing liquid is reboiled. An embodiment of the invention is one, any or all of prior embodiments in this paragraph up through the first embodiment in this paragraph, wherein the ethylene stream comprises a portion of a dehydrated effluent. An embodiment of the invention is one, any or all of prior embodiments in this paragraph up through the first embodiment in this paragraph, wherein the ethylene stream has a pressure between 3,447 to 4,137 kPa (500 to 600 psi(g)) and the liquid stream has a pressure between 2,413 to 3,103 kPa (350 to 500 psi(g)). An embodiment of the invention is one, any or all of prior embodiments in this paragraph up through the first embodiment in this paragraph further comprising recovering power from a pressure decrease from the ethylene depleted vapor with an expansion device. An embodiment of the invention is one, any or all of prior embodiments in this paragraph up through the first embodiment in this paragraph further comprising cooling a process stream with the liquid stream. An embodiment of the invention is one, any or all of prior embodiments in this paragraph up through the first embodiment in this paragraph further comprising separating the liquid ethylene stream in a oxygenate fractionation zone comprising a fractionation column into a liquid stream comprising oxygenates and a oxygenate fractionation zone overhead stream; separating the oxygenate fractionation zone overhead stream in a vessel into a liquid portion and a gaseous portion, wherein the gaseous portion comprises ethylene and carbon dioxide. An embodiment of the invention is one, any or all of prior embodiments in this paragraph up through the first embodiment in this paragraph further comprising reboiling a stream from the fractionation column in the oxygenate fractionation zone with a hot process stream. An embodiment of the invention is one, any or all of prior embodiments in this paragraph up through the first embodiment in this paragraph further comprising cooling the oxygenate fractionation zone overhead stream with a refrigerant stream in a cooling zone before separating the overhead stream in the vessel.

A second embodiment of the invention is a process for producing jet range hydrocarbons from a biobased alcohol comprising dehydrating a biobased alcohol stream in a dehydration zone comprising a reactor with a catalyst and being operated under conditions to provide a dehydrated effluent, the dehydrated effluent comprising ethylene and carbon monoxide; separating, from a feed stream comprising a portion of the dehydrated effluent, the carbon monoxide from the ethylene in a fractionation zone, the fractionation zone providing a bottoms stream comprising a carbon monoxide depleted ethylene stream and an overhead stream comprising carbon monoxide and ethylene; absorbing ethylene from a portion of the overhead stream in an absorption zone with an absorbing liquid stream to provide an enriched absorbing liquid stream comprising an increased level of ethylene and an ethylene depleted vapor stream comprising carbon monoxide; oligomerizing the carbon monoxide depleted liquid ethylene stream in an oligomerizing zone comprising a reactor with a catalyst and being operated under conditions to provide an oligomerized effluent; hydrogenating the oligomerized effluent in a hydrogenation zone having a hydrogenation reactor with a catalyst and being operated under conditions to provide a hydrogenated effluent; and, separating the hydrogenated effluent into one or more hydrocarbon streams, the one or more hydrocarbon streams comprising a jet fuel hydrocarbon steam. An embodiment of the invention is one, any or all of prior embodiments in this paragraph up through the second embodiment in this paragraph, wherein the absorbing liquid stream comprises the oligomerized effluent. An embodiment of the invention is one, any or all of prior embodiments in this paragraph up through the second embodiment in this paragraph, further comprising separating the overhead stream in a vessel into a liquid portion and a gaseous portion, wherein the gaseous portion comprises the portion of the overhead stream from which ethylene is absorbed in the absorption zone; or cooling the overhead stream with a refrigerant stream in a cooling zone before separating the overhead stream; or both. An embodiment of the invention is one, any or all of prior embodiments in this paragraph up through the second embodiment in this paragraph, further comprising separating the oxygenates from the carbon monoxide depleted ethylene stream in a oxygenate fractionation zone comprising a fractionation column into a liquid stream comprising oxygenates and a oxygenate fractionation zone overhead stream before oligomerizing the carbon monoxide depleted ethylene stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for separating carbon monoxide from ethylene, the process comprising:
cooling an ethylene stream with a refrigerant stream in a cooling zone;
separating the ethylene stream in a fractionation zone comprising a fractionation column into a liquid stream comprising ethylene and an overhead stream comprising carbon monoxide and ethylene;
separating the overhead stream in a vessel into a liquid portion and a gaseous portion, wherein the gaseous portion comprises carbon monoxide and ethylene; and,
absorbing the ethylene from the gaseous portion in an absorption zone with an absorbing liquid stream to provide an enriched absorbing liquid comprising an increased level of ethylene and an ethylene depleted vapor comprising carbon monoxide.

2. The process of claim 1, further comprising:
reboiling a stream from the fractionation column with the ethylene stream before cooling the ethylene stream in the cooling zone.

3. The process of claim 1, further comprising:
heating the liquid stream from the fractionation column with the ethylene stream before cooling the ethylene stream in the cooling zone.

4. The process of claim 1, further comprising:
cooling the overhead stream with a refrigerant stream in a cooling zone before separating the overhead stream in a vessel.

5. The process of claim 1, further comprising:
subcooling the refrigerant stream by transferring heat from the refrigerant stream to a portion of the liquid stream from the fractionation column.

6. The process of claim 1, further comprising:
subcooling the refrigerant stream by transferring heat from the refrigerant stream to the gaseous portion from the vessel.

7. The process of claim 1, wherein the absorbing liquid stream comprises a hydrocarbon stream.

8. The process of claim 7, wherein the hydrocarbon stream comprises an oligomerized effluent.

9. The process of claim 7, wherein the enriched absorbing liquid is reboiled.

10. The process of claim 1, wherein the ethylene stream comprises a portion of a dehydrated effluent.

11. The process of claim 1, wherein the ethylene stream has a pressure between 3,447 to 4,137 kPa (500 to 600 psi(g)) and the liquid stream has a pressure between 2,413 to 3,103 kPa (350 to 500 psi(g)).

12. The process of claim 1 further comprising:
recovering power from a pressure decrease from the ethylene depleted vapor with an expansion device.

13. The process of claim 1 further comprising:
cooling a process stream with the liquid stream.

14. The process of claim 1 further comprising:
separating the liquid stream comprising ethylene in a oxygenate fractionation zone comprising a fractionation column into a liquid stream comprising oxygenates and a oxygenate fractionation zone overhead stream;
separating the oxygenate fractionation zone overhead stream in a vessel into a liquid portion and a gaseous portion, wherein the gaseous portion comprises ethylene and carbon dioxide.

15. The process of claim 14 further comprising:
reboiling a stream from the fractionation column in the oxygenate fractionation zone with a hot process stream.

16. The process of claim 14 further comprising:
cooling the oxygenate fractionation zone overhead stream with a refrigerant stream in a cooling zone before separating the overhead stream in the vessel.

17. A process for producing jet range hydrocarbons from a biobased alcohol comprising:
dehydrating a biobased alcohol stream in a dehydration zone comprising a reactor with a catalyst and being operated under conditions to provide a dehydrated effluent, the dehydrated effluent comprising ethylene and carbon monoxide;
separating, from a feed stream comprising a portion of the dehydrated effluent, the carbon monoxide from the ethylene in a fractionation zone, the fractionation zone providing a bottoms stream comprising a carbon monoxide depleted ethylene stream and an overhead stream comprising carbon monoxide and ethylene;
absorbing ethylene from a portion of the overhead stream in an absorption zone with an absorbing liquid stream to provide an enriched absorbing liquid stream comprising an increased level of ethylene and an ethylene depleted vapor stream comprising carbon monoxide;
oligomerizing the carbon monoxide depleted liquid ethylene stream in an oligomerizing zone comprising a reactor with a catalyst and being operated under conditions to provide an oligomerized effluent;
hydrogenating the oligomerized effluent in a hydrogenation zone having a hydrogenation reactor with a catalyst and being operated under conditions to provide a hydrogenated effluent; and,
separating the hydrogenated effluent into one or more hydrocarbon streams, the one or more hydrocarbon streams comprising a jet fuel hydrocarbon steam.

18. The process of claim 17, wherein the absorbing liquid stream comprises the oligomerized effluent.

19. The process of claim 17, further comprising:
separating the overhead stream in a vessel into a liquid portion and a gaseous portion, wherein the gaseous portion comprises the portion of the overhead stream from which ethylene is absorbed in the absorption zone; or
cooling the overhead stream with a refrigerant stream in a cooling zone before separating the overhead stream; or
both.

20. The process of claim 17, further comprising:
separating oxygenates from the carbon monoxide depleted ethylene stream in a oxygenate fractionation zone comprising a fractionation column into a liquid stream comprising oxygenates and a oxygenate fractionation zone overhead stream before oligomerizing the carbon monoxide depleted ethylene stream.
